(19) United States
(12) Patent Application Publication
(10) Pub. No.: US 2008/0319269 A1
(43) Pub. Date: Dec. 25, 2008

(54) Rectally Insertable Surgical System

(76) Inventors: Antonio Longo, Palermo (IT); Jesse J. Kuhns, Cincinnati, OH (US); Michele D'Arcangelo, Roma (IT); Federico Bilotti, Aprilia (IT)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/997,960

(22) PCT Filed: Aug. 9, 2005
(86) PCT No.: PCT/EP2005/008656

§ 371 Date: Jul. 23, 2008

(51) Int. Cl. A61B 1/32

(52) U.S. Cl. 600/235

(57) ABSTRACT

In a rectally insertable surgical system (1), a sleeve device (2) having a proximal end and a distal end (16) is open at its proximal end and has a window area (20) longitudinally extending on one side of the sleeve device (2) up to its distal end region. A shield (4) is adapted to slide along the sleeve device (2) to at least partially close the window area (20). The sleeve device (2) including the shield (4) is insertable into an anus dilator ring (6).

The invention relates to a rectally insertable surgical system, which can be used, e.g., during a so called PPH surgical procedure (“Procedure for Prolapse and Haemorrhoids”).

The PPH technique was first introduced in Italy in 1997 and was introduced in the United States of America in October 2001. During a PPH procedure, haemorrhoidal tissue is caught by means of a purse string suture and excised. To this end, an anus dilator ring accommodating a sleeve device having an atraumatic tip region is inserted into the anus and the rectum. Once the anus dilator ring has been placed, the sleeve device can be retracted, if required. The sleeve device provides a working channel and, because of a lateral window extending longitudinally on one side, allows access to the site of surgery in order to place the suture.

A disadvantage of the known sleeve device is the tendency of tissue, in particular protruding haemorrhoidal tissue, to fall, through part of the lateral window area, into the working channel provided by the sleeve device. This leads to the surgeon's view being limited and altered. It further limits the amount of space available to do work, and it can also lead to potential damaging tissue structures.

The object of the present invention is to provide a possibility to reduce these drawbacks.

This problem is solved by a rectally insertable surgical system having the features of claim 1. Advantageous versions of the invention follow from the dependent claims.

The rectally insertable surgical system according to the invention comprises a sleeve device, a shield, and an anus dilator ring. The sleeve device has a proximal end and a distal end and is open at its proximal end. Moreover, it has a window area longitudinally extending on one side of the sleeve device up to its distal end region. The shield is adapted to slide along the sleeve device to at least partially close the window area. The sleeve device including the shield are insertable into the anus dilator ring.

According to the invention, the shield can be placed by sliding along the sleeve device to a position such that tissue is prevented from falling into the working channel provided by the sleeve device. Thus, the amount of working space available at the window area of the sleeve device is increased, which facilitates the surgical procedure, e.g., a purse-string suture can be positioned and placed in a more uniform fashion around the rectal walls.

Preferably, the sleeve device comprises a half-sleeve which is open at its proximal end, wherein the window area extends longitudinally on one side of the half-sleeve from its proximal end up to its distal end region. Roughly speaking, in this design the sleeve device has a cross-sectional shape with a periphery extending over an arc in the order of 180°. However, the term “half-sleeve” includes arc angles different from 180° as well and is not restricted to an angle corresponding to exactly one half of a full circle. Such design provides a large window area. In a preferred embodiment, the shield, in the area where it closes the window area, completes the cross-section of the half-sleeve to that of a full-sleeve, preferably to that of one having a circular cross-sectional shape. That means the sleeve device including the shield acts like a full-sleeve providing a rigid working channel when the shield has been moved along the sleeve device to its distal position. In this state, the system can already provide an open window area when the shield does not extend along the total length of the sleeve device. In any case, the size of the window area can be adapted to the desired one when the shield is moved in proximal direction.

The sleeve device can comprise an atraumatically shaped tip at its distal end. Preferably, the tip has a half-conical shape, e.g., when a sleeve device designed as a half-sleeve extends to a conical tip area. This design provides for a large window area in order to facilitate access to the tissue to be handled during the surgery.

In a preferred version of the surgical system according to the invention, the sleeve device has at least one recess in its wall close to its distal end and preferably has two such recesses at positions approximately diametrically opposite to each other. These recesses increase the access space for handling the tissue, in particular during the placement of a purse-string suture.

Preferably, the shield is removable from the sleeve device. In this way, the shield can be attached before the procedure or during the procedure, and the system can be used without a shield as a conventional system having a sleeve device and an anus dilator ring only.

The shape of the distal end side of the shield can vary according to the specific needs of the surgery in question. For example, the shield can have a straight edge (which is curved in circumferential direction only) but it can also have a rounded distal end side, i.e. an end side which varies in longitudinal direction of the shield. The end side of the shield can have a convex curvature, which particularly minimizes trauma to the tissue upon insertion. Another possibility is a concave shape which provides a convenient rounded periphery of the window area when the shield has been moved to its desired position.

Preferably, the shield has a grip in its proximal end region. This grip serves as a handle and can also serve as a stop when the shield has been moved to its most distal position. The shield can have various types of handle configurations (designed as, e.g., a rounded lip, a tab, an extend handle, a recessed area) that allow the surgeon to be able to change the location of the shield at any time (prior to insertion of the sleeve device, after insertion of the sleeve device, or for removal of the sleeve device).

In preferred embodiments of the invention, the sleeve device, at the longitudinal edges of the window area, comprises two guide rails adapted to slidingly guide the shield. Preferably, the guide rails are designed such that the shield is removable and can be laterally put onto the guide rails. In this case, the shield can be laterally secured by the anus dilator ring when the system is assembled. This enables a space-saving design of the guide rails because the shield does not have to be secured against lateral detachment by means of the guide rails as this function is provided by the anus dilator ring. Alternatively, the guide rails can secure the shield against removal in lateral or transversal direction, which, however, generally requires a more space-consuming design of the guide rails which might decrease the size of the working space inside the sleeve device.

Preferably, the anus dilator ring comprises a sleeve having inner dimensions adapted to the outer dimensions of the sleeve device including the shield. The anus dilator ring can be provided with at least one grip, which preferably transversely extends from its proximal end region. Moreover, the sleeve device preferably comprises a grip as well, which can extend transversely from its proximal end region.

As an alternative to a tip at its distal end, the sleeve device can be open at its distal end. This facilitates the use of surgical instruments which have to be forwarded to a position deeper than the anus, e.g., a curved stapler and cutter (see, e.g., DE 100 26 683 A1) used for the treatment of so called ODS causing pathologies (e.g. rectal prolapse, rectocele, intussusception). In order to insert such a sleeve device to its desired position, the use of an atraumatic auxiliary means placed inside the sleeve might be helpful, which is retracted after insertion of the sleeve device.

Other pathologies that could be treated with the same approach are deep lesions (small tumors and polyps) in the rectum. Technology already exists for treatment of rectal pathologies such as tumors and polyps, as for example transanal endoscopic microsurgery (TEM). In this technique, the surgeon performs the operation through a scope placed into the anal canal. A smaller telescope is used to magnify the tumor. TEM incorporates scopes for viewing, surgical instruments and a device to dilate the rectum with gas to perform the surgery through the anus. This means that patients may be able to avoid traditional, open surgery and an abdominal incision. This will allow them to resume normal activities much sooner. The greatest benefit of TEM is that a patient can avoid open surgery and experience less pain and time recovering from surgery. Patients can often return to normal activity sooner and experience little to no pain after surgery. Sometimes, as a result of open surgery, a patient may need a colostomy (the surgical construction of an artificial anus between the colon and the surface of the abdomen). Another benefit of TEM is that the need for a colostomy is rare and unlikely. Other risks associated with an abdominal incision are avoided, such as wound infection, and pulmonary infection.

There are several benefits of using a curved stapler and cutter with the current invention, over the TEM technique, for the treatment of deep rectal lesions (small tumors and polyps) in the rectum: (1) Less invasive because of overall smaller diameter (TEM requires 40 mm) and therefore less risk of patient incontinence; (2) use of endoluminal surgical stapler versus conventional surgical instruments (i.e. full wall excision, better hemostasis, transverse staple line that is not subject to stenosis); (3) much simpler and less time consuming (only one surgical stapler versus several surgical instruments).

In the following, the rectally insertable surgical system and its application in surgery are explained in more detail by means of a preferred embodiment. The drawings show in

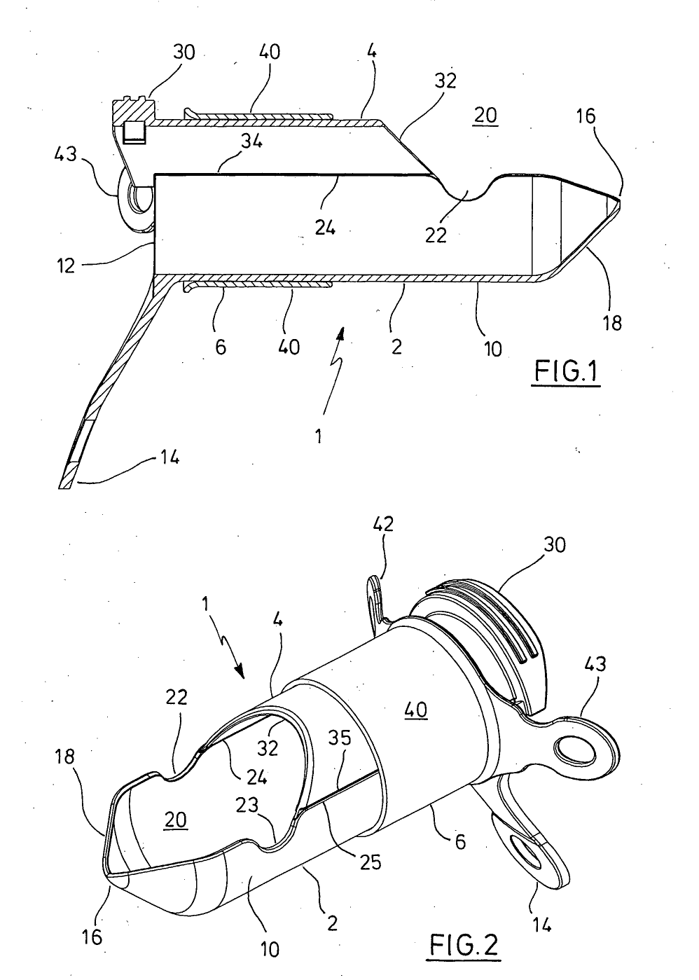

FIG. 1 a side view of a preferred embodiment of the surgical system according to the invention in the assembled state, some parts shown in longitudinal section in the longitudinal center plane of the system as well,

FIG. 2 an isometric view of the surgical system of FIG. 1 in the assembled state, including a sleeve device, a shield, and an anus dilator ring,

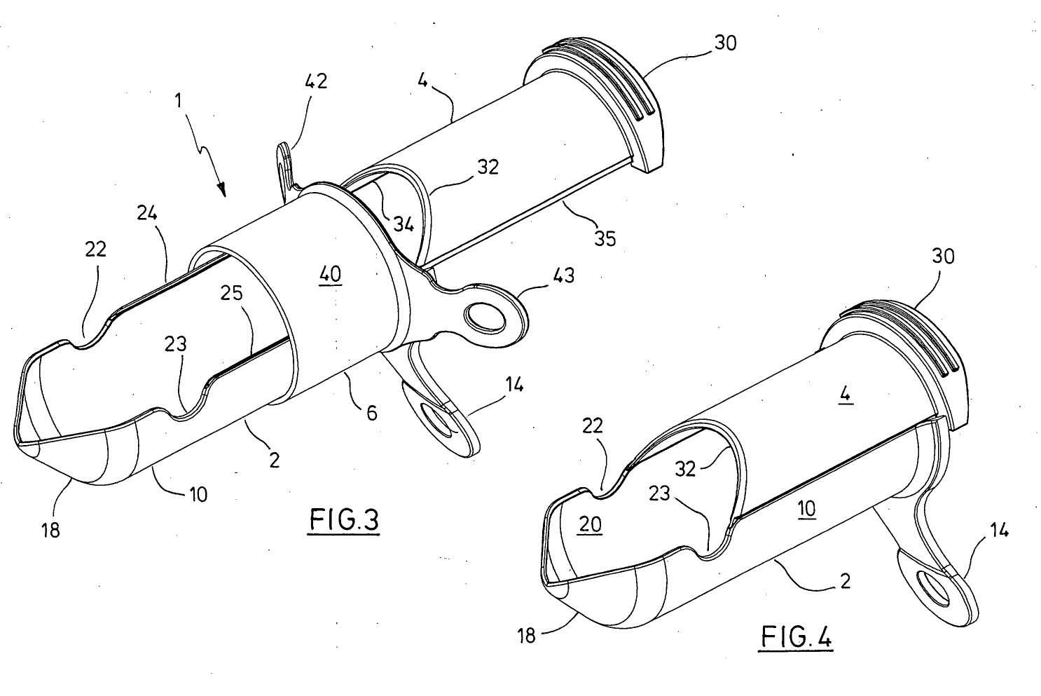

FIG. 3 an isometric view similar to FIG. 2, the shield having been retracted,

FIG. 4 an isometric view similar to FIG. 2, the anus dilator ring having been removed,

FIG. 5 an isometric view of the anus dilator ring of the embodiment of the surgical system, and

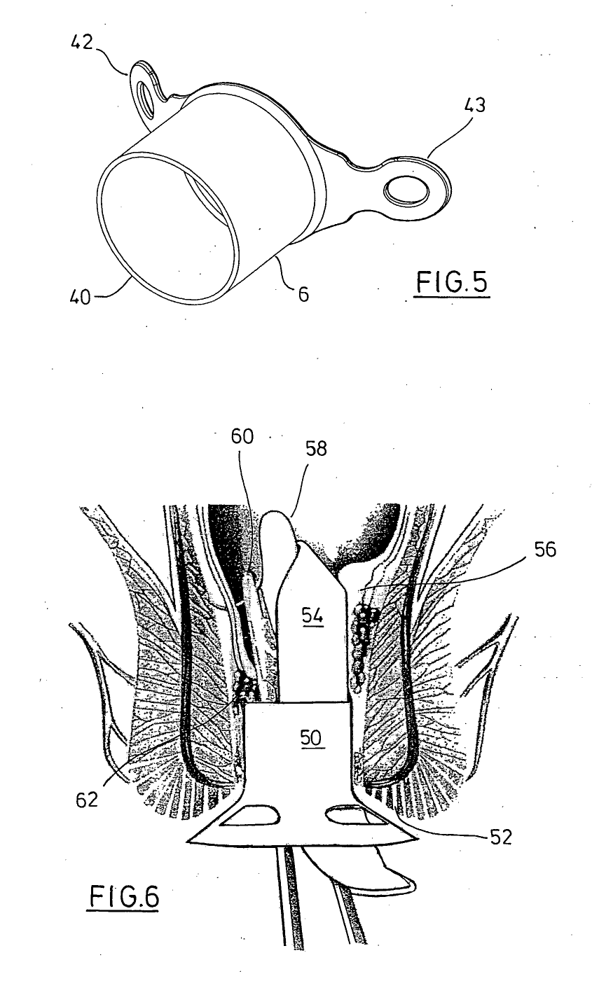

FIG. 6 a schematic representation of a prior art sleeve device and anus dilator ring in use during a PPH procedure.

FIG. 1 displays a side view of a preferred embodiment of the surgical system, here designated by reference numeral 1. It also shows a longitudinal section of the surgical system 1 in the longitudinal center plane. In FIG. 1, the surgical system 1 is in the assembled state. It comprises a sleeve device 2, a shield 4 and an anus dilator ring 6.

As illustrated in the isometric views of FIGS. 2, 3 and 4, the sleeve device 2 is designed as a half-sleeve 10, i.e. in cross-section, the half-sleeve 10 extends about an angle in the order of 180°. This angle may vary, however, in different designs of the half-sleeve.

The sleeve device 2 has a proximal end 12, which is open in order to allow free access to the interior of the sleeve device 2. At the proximal end 12 a grip 14 is attached to the half-sleeve 10 in order to facilitate its handling. At its distal end 16, the sleeve device 2 is provided with a half-conical nose 18. The nose 18 is atraumatically shaped.

The sleeve device 2 has a window area 20, which longitudinally extends on one side of the sleeve device 2. Generally, the window area 20 is provided by the missing half of the half-sleeve 10 which is not blocked by the anus dilator ring 6. The size of the window area 20 can be adapted by means of the shield 4, as is explained in more detail further below. On both lateral sides, the window area 20 is increased by recesses 22 and 23 which can be particularly useful, e.g., for guiding a needle holder when a purse-string suture is made. At the longitudinal edges of the window area 20, the sleeve device 2 comprises guide rails 24 and 25 for slidingly guiding the shield 4 (see below).

In the cross-section of the assembled surgical system 1, the shield 4 completes the circular shape of the half-sleeve 10, see FIGS. 2 and 4. In its proximal end area, a shield 4 comprises a grip 30 which also serves as a stop. The distal end side of the shield 4 has a concavely rounded shape 32. The longitudinal edges 34 and 35 of the shield 4 are provided with guide rails which match to their counterparts 24 and 25 of the sleeve device 2.

In order to assemble the sleeve device 2 and the shield 4, the shield 4 is laterally attached to the sleeve device 2 such that the configuration shown in FIG. 4 is achieved. In the embodiment, the design of the guide rails 24 and 25 of the sleeve device 2 and of the longitudinal edges 34 and 35 of the shield 4 allow for such positioning. In the state shown in FIG. 4, the shield 4 is not secured against lateral removal, but in the fully assembled state of the surgical system 1, the anus dilator ring 6 secures and guides the shield 4. The advantage of this design of the guide rails 24 and 25 and the longitudinal edges 34 and 35, which does not provide for a retaining means against lateral removal in the guide rail system, is a simple and reliable set-up without protruding parts (which would decrease the free cross-sectional area of the sleeve device 2) and the ability to quickly attach the shield 4 to the sleeve device 2 from the side.

The anus dilator ring 6 is shown in FIG. 5. It comprises a sleeve 40 and two grips 42 and 43 extending from the sleeve 40 in diametrically opposite directions. The inner diameter of sleeve 40 matches to the outer diameter of the half-sleeve 10 when the shield 4 is inserted.

In use, the size of the window area 20 can be adjusted by sliding the shield 4 to the desired position. The shield 4 is easily accessible via its grip 30. It is possible to completely remove the shield 4 by pulling at grip 30. Moreover, the shield 4 can be inserted from the proximal end sides of the sleeve device 2 and the anus dilator ring 6, if required during a surgical procedure.

The surgical system 1 is used similarly to the prior art devices, but has the advantage that the size of the window area 20 can be adjusted during the surgical procedure. This is explained by means of FIG. 6, which shows a conventional anus dilator 50 inserted in the anus 52 of a patient and a conventional sleeve device 54, which is inserted through the anus dilator 50 up to the patient's rectum 56.

FIG. 6 illustrates how a purse-string suture 58 is laid by means of a surgical needle holder 60 in a PPH procedure. In such a procedure, haemorrhoidal tissue 62 is caught by the purse-string suture 58 and can be resected afterwards by a cutter which is introduced into the working channel provided by the anus dilator 50 and the sleeve device 54.

As said, FIG. 6 displays the conditions when a conventional sleeve device 54 is used. In this case, haemorrhoidal tissue 62 marked by an oval line is present in or enters into the working area, blocks the surgeon's view, limits the amount of space available to do work and is susceptible to damage. In order to avoid all these problems, the surgical system 1 described by means of FIGS. 1 to 5 comprises the shield 4 which is moved to a desired position in order to adjust the size of the window area 20 such that tissue like haemorrhoidal tissue 62 cannot enter the working space.

Thus, the surgical system 1 is generally used like a conventional system. It can even be applied without the shield 4 in which case it has the same properties as a conventional system. The shield 4, however, allows for an adjustment of the window area 20 which largely facilitates the surgical procedure.

In the embodiment explained so far, the sleeve device of the system has an atraumatically shaped tip (nose 18) at its distal end. In other embodiments, the sleeve device is open at its distal end, but otherwise similarly shaped. Such design can be used for the treatment of ODS causing pathologies and deep lesions, as discussed above.

1. Rectally insertable surgical system, comprising
a sleeve device (2) which has a proximal end (12) and a distal end (16) and which is open at its proximal end (12) and has a window area (20) longitudinally extending on one side of the sleeve device (2) up to its distal end region,
a shield (4) which is adapted to slide along the sleeve device (2) to at least partially close the window area (20), and
an anus dilator ring (6) into which the sleeve device (2) including the shield (4) is insertable.

2. Surgical system according to claim 1, characterized in that the sleeve device (2) comprises a half-sleeve (10) which is open at its proximal end (12), wherein the window area (20) extends longitudinally on one side of the half-sleeve (10) from its proximal end (12) up to its distal end region.

3. Surgical system according to claim 2, characterized in that the shield (4), in the area where it closes the window area (20), completes the cross-section of the half-sleeve (10) to that of a full-sleeve, preferably to that of one having a circular cross-sectional shape.

4. Surgical system according to one of claims 1 to 3, characterized in that the sleeve device (2) comprises an atraumatically shaped tip at its distal end (16), the tip preferably having a half-conical shape (18).

5. Surgical system according to one of claims 1 to 3, characterized in that the sleeve device is open at its distal end.

6. Surgical system according to one of claims 1 to 5, characterized in that the sleeve device (2) has at least one recess (22, 23) in its wall close to its distal end (16).

7. Surgical system according to claim 6, characterized in that the sleeve device (2) has two approximately diametrically opposite recesses (22, 23) close to its distal end (16).

8. Surgical system according to one of claims 1 to 7, characterized in that the shield (4) is removable.

9. Surgical system according to one of claims 1 to 8, characterized in that the shield (4) has a rounded distal end side (32).

10. Surgical system according to one of claims 1 to 9, characterized in that the shield (4) has a grip (30) in its proximal end region.

11. Surgical system according to one of claims 1 to 10, characterized in that the sleeve device (2), at the longitudinal edges of the window area (20), comprises two guide rails (24, 25) adapted to slidingly guide the shield (4).

12. Surgical system according to claim 11, characterized in that the shield (4) is removable and can be laterally put onto the guide rails (24, 25).

13. Surgical system according to claim 12, characterized in that the shield (4), when the system (1) is assembled, is laterally secured by the anus dilator ring (6).

14. Surgical system according to one of claims 1 to 13, characterized in that the anus dilator ring (6) comprises a sleeve (40) having inner dimensions adapted to the outer dimensions of the sleeve device (2) including the shield (4).

15. Surgical system according to one of claims 1 to 14, characterized in that the sleeve device (2) comprises a grip (14), which preferably transversely extends from its proximal end region.

16. Surgical system according to one of claims 1 to 15, characterized in that the anus dilator ring (6) is provided with at least one grip (42, 43), which preferably transversely extends from its proximal end region.
